(19) United States
(12) Patent Application Publication
(10) Pub. No.: US 2012/0191164 A1
(43) Pub. Date: Jul. 26, 2012

(54) Radiant heating apparatus and method for therapeutic heating

(76) Inventors: Nicholas M. Gander, East Sussex (GB); Brian G. Howard, Hampshire (GB)

(21) Appl. No.: 12/931,360

(22) Filed: Jan. 26, 2011

(51) Int. Cl. A61F 7/08; H05B 3/36; H05B 3/34

(52) U.S. Cl. 607/96; 219/528

(57) ABSTRACT

The present invention provides a method and apparatus for heating via infrared radiation. The invention may incorporate a heating pad, safe to the touch, for therapeutic treatment. A heating pad according to the present invention may comprise of layers, such as a heat generation layer, electrically insulating layers, radiation permeable layers, with a sealed cover enclosing the entire pad. This invention is especially useful for heat therapy when a heat pad is applied to the skin of a human or an animal.

### BACKGROUND OF THE INVENTION

The present invention relates generally to an improved heating apparatus that is beneficial for therapeutic use where an antibacterial or antiviral environment is desired plus other heating uses and more particularly to an improved multi-flexible radiant heating apparatus approaching a final surface temperature no greater than the body temperature of the user while achieving enhanced penetrative warmth.

Heat is applied therapeutically to body parts to treat injuries, soreness, pain, arthritis, bursitis, and other ailments. Past heating methods employ heating pads, but fail to adequately control the production of heat via electrical sources, such that excessive use can burn or otherwise injure or cause unnecessary pain. Additionally, ordinary heating pads lack materials and design adapted to properly control the heat applied to body or skin surfaces or to provide adequate heat while avoiding excessive convection heat damage. For example, prior heating apparatuses fail to provide an enhanced therapeutic effect without encountering the risk of hot or burning feelings on the skin of the user.

Prior art heating pads are generally comprised of insulated electrical heating elements held within a sealed bag, covered with a washable removable cloth envelope. Such heating pads are resistance heated by the flow of electricity there through, which heats the surrounding insulated envelope. The pad is applied to an area of the body which is to be treated, and the hot pad provides fairly uneven heat to the skin.

However the use of this type of heating pad must be carefully controlled. Since the pad heats the skin by conduction of heat emanating from the heating coils and to the body of the user, the pad generally feels hot to the touch. Also, use of the heating pad must be limited or the skin can be burned, particularly if the user falls asleep while using the pad. Due to the conduction of heat to the skin, the pad eventually begins feeling very uncomfortable. It is now recognized that this type of heat application can cause detrimental effects within veins, capillaries, and arteries of the circulatory system causing blood vessels to become engorged and breakdown particularly, within the paws of animals. While such pads generally use thermostats to control the amount of heat generated, using the heating pad in a confined space, such as under the patient/animal, generally causes an accumulation of heat directly on the skin which is conducted directly from the heating coils. This heat has been found to eventually become uncomfortable even at a generally low thermostatic setting. When in use with animals under the influence of anesthesia or children under the age of about six where body temperature regulating mechanisms are not fully functioning, detrimental responses may be observed particularly with a local, conducted uneven heat source of this type.

Some heating schemes exist employing a therapeutic heating pad which operates using black body radiation of infrared heat, rather than conduction heating. The surface temperature of such heating pads may easily exceed the temperature of the human body, somewhat, but because of the design the heat is dissipated and it does not feel uncomfortably hot to the touch. Thus such a heating pad may be used for extended periods of time. However the radiant heat generated by the heating pad has been found to penetrate the tissues relatively deeply, thus providing an enhanced therapeutic effect. However many carbon heat panel systems are set on a rigid/solid base and or sometimes a rigid protection cover with perhaps a flexibility in only one direction. Such limited flexibility may cause other problems, such as localized pressure points to the animal and or human individual.

As can be seen, there is a need for an improved heating apparatus and methods for supplying heat to solve the problem of lack of flexibility, insufficient heat penetration of tissues, and excessive heat damage to the skin and other tissues of the user, for example, a method that radiates heat that is well controlled.

### SUMMARY OF THE INVENTION

In one aspect of the present invention, a heating pad comprises woven radiant heating strips; an electrically conductive ribbon electrically connected to the radiant heating strips; a pair of isolator layers, each isolator layer adjacent to one side of the radiant heating strips; and a thermally reflective layer situated between a side of the radiant heating strips and a proximal side of an enveloping cover; wherein the enveloping cover is adapted to enclose the woven radiant heating strips, the electrically conductive ribbon, the pair of isolator layers, and the thermally reflective layer.

In another aspect of the present invention, a heat generating apparatus comprises a heating pad further comprising woven radiant heating strips; an electrically conductive ribbon electrically connected to the radiant heating strips; an electrical wire adapted to transmit electrical current to the electrically conductive ribbons; a thermally reflective layer situated between a side of the radiant heating strips and a proximal side of an enveloping cover; and a flexible envelope of radiation permeable material enclosing the radiant heating strips and adapted such the electrical wire extends out of the flexible envelope.

These and other aspects, objects, features and advantages of the present invention, are specifically set forth in, or will become apparent from, the following detailed description of an exemplary embodiment of the invention when read in conjunction with the accompanying drawings.

### BRIEF DESCRIPTION OF THE DRAWINGS

FIG. 1 is a perspective view of complete heating apparatus, according to an embodiment of the present invention;

FIG. 2 is a side view of a heating pad in use, according to an embodiment of the present invention;

FIG. 3 is a cross-sectional view of a heating apparatus, according to an embodiment of the present invention; and

FIG. 4 is a plan view of a heating apparatus, with the layers removed except for the heat generating layer, according to another embodiment of the present invention.

### DETAILED DESCRIPTION OF THE INVENTION

The following detailed description is of the best currently contemplated modes of carrying out the invention. The description is not to be taken in a limiting sense, but is made merely for the purpose of illustrating the general principles of the invention, since the scope of the invention is best defined by the appended claims.

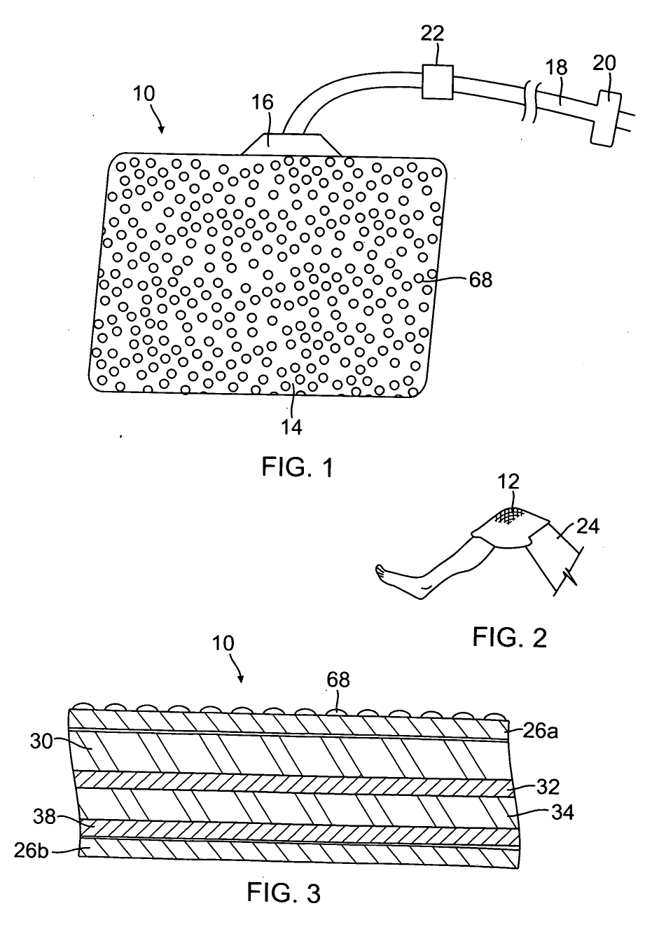

Referring now to the drawings in detail, wherein like reference characters refer to like elements, there is shown in FIG. 1 a perspective view of a heating apparatus, featuring a heating pad 10. The heating pad 10 may be manufactured having a sealed outer membrane which may be made of synthetic or other suitable materials to prevent the ingress of foreign matter and moisture. Cover 14 which may be manufactured of cloth containing cotton, bamboo, other natural fiber, synthetic materials, and/or blends thereof and closed by a zipper, a hook and loop fastener (Velcro), or other suitable fastener. An electrical cord 18 may be used for carrying current to the heating pad 10 which may be controlled by an inline cord on-off switch 22. The electrical cord 18 may extend from an opening 16 which may be heat sealed closed, or by another suitable closure means. The on-off switch 22 may incorporate a variable temperature control. It should be noted that the AC main electrical plug 20 shown at the end of power cord 18, upon appropriate design of the heating pad 10, various potentials can be used, such as 220 volts, 240 volts, 110 volts, 24 volts, 12 volts, 75 volts, and the like, whether alternating current (AC) or direct current (DC). A source of electrical power may be a re-chargeable battery pack (not shown) for enhanced portability of the heating pad 10.

The surface temperature of the heating pad 10 may be limited to reach no higher than about 30 degrees Celsius to about 34 degrees Celsius (about 86 degrees Fahrenheit to about 93.2 degrees Fahrenheit) and often on the order of about 38 degrees Celsius (about 100.4 degrees Fahrenheit). Infrared radiation provided by the heating pad 10 of the present invention may penetrate deeply into the body of a user and especially into muscle of the user for the desired therapeutic benefit of the heating pad 10. The wavelength range for far infrared radiation may be from about 5 to about 25 microns, and often ranging from about 6 microns to about 15 microns. With the above advantages, the heating pad 10 may be used in place for even more extended periods of time with a comfortably warm sensation, but without causing severe skin surface burns and achieving an improved penetrative warmth without overheating the subject.

FIG. 2 shows a side view of a heating pad 12 in use placed over the knee joint of a leg 24 of the user. The surface temperature of the pad 10 may range about 30 degrees Celsius to about 38 degrees Celsius (about 86 degrees Fahrenheit to about 100.4 degrees Fahrenheit), and consequently the pad may be used in place for a long period of time while maintaining a comfortably warm sensation. Yet the infrared radiation may penetrate deeply into the body, imparting maximum therapeutic effect by delivering deep heat without causing skin burns.

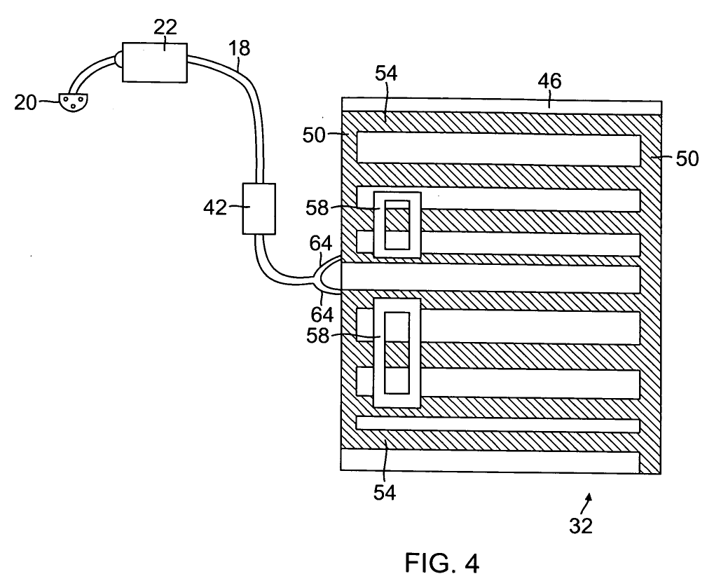

FIG. 3 shows a cross section of a portion of the heating apparatus 10. The heat generating layer 32 may be sandwiched by a pair of isolator layers, such that each isolator layer 30,34 is adjacent to one side of the radiant heating strips 54 (shown in FIG. 4), covering the surface of the heat generating layer 32 on opposite sides. The isolator layers 30, 34 may be foam, such as a black sponge layer. The color black for the sponge material may be useful to enhance black body radiation. For example, an outer isolator layer 30 may be placed on top of the heat generating layer 32 and an inner isolator layer 34 may be placed below the heat generating layer 32. The isolator layers 30, 34 may be insulative to electricity, but conductive to infrared radiation.

A reflective layer 38 may be disposed against the lower surface of the isolator layer 34. The reflective layer 38 may be made from aluminum foil of about 0.02 mil (2×10⁻⁵ inches; 5.08×10⁻⁵ centimeters) thickness with a polyester coating. However other types of reflective materials may be used if suitably reflective.

An outer enveloping layer 26 (such as comprising layers 26a and 26b) may enclose isolator layers 30,34 to enclose the entire heating apparatus 10. This enveloping layer 26a-26b may be manufactured of vinyl or other suitable material (polymers, bamboo material, and the like), may holds all of the above described layers in a laminated position and keeps the layers dry and protects against contaminant intrusion.

The outer enveloping layer 26a-26b may be situated inside an outer cloth bag. The cloth bag may be made of terry towel, or other cotton or natural fiber material (such as bamboo), or synthetic materials. Any suitable material shown to be comfortable to the user is desirable. The cloth bag may be closed by a zipper or with other fastening devices such as closures, snaps, or the like. The cloth bag may be removed and washed as desired. This may include an additional vibrating mechanism.

The heating pad 10 may be plugged in, or otherwise connected to a source of operating current. The heating pad 12 may be placed over a region to be therapeutically warmed with deep heat, such as shown in FIG. 2. Current passes through the resistive heating strips 54 (shown in FIG. 4), creating a source of black-body infrared radiation. The radiation may be received by the body of the user, penetrate deeply into the user's body. Still, the heating pad 10 does not feel uncomfortably hot to the touch.

FIG. 4 shows a plan view of a heating apparatus, with the layers removed except for the heat generating layer 32, according to another embodiment of the present invention. The heat generating layer 32 may comprise a material made from carbon powder, such as carbon fiber material manufactured by Toray Industries, Inc., located in Nihonbashi Mitsui Tower, 1-1, Nihonbashi-Muromachi 2-chome, Chuo-ku, Tokyo 103-8666, Japan. For example, a carbon fiber material as described in U.S. Pat. No. 4,983,814 (the contents of which are incorporated herein) may be used.

It should be noted that materials other than the carbon fiber described above may be used as a base for the resistive material. For example, as described in U.S. Pat. No. 3,865,626 (the contents of which are incorporated herein), a polyester film may be treated with a solvent or swelling agent. Electroconductive particles, preferably carbon black, may then be applied to the treated surface in a concentration corresponding to the desired resistance. The film may then be subjected to heat treatment to solidify and retain the resistive material on the surface. Since the resistance material is basically fabricated of carbon compound, the heating strip 54 is a black body radiator, which has a high efficiency of radiant heat dispersion which, contributes to the effectiveness of the invention.

Conductive ribbons 50 may be sewn or otherwise adhered into electrical contact with the heating strips 54, optionally along parallel edges of the heat generator 46. The ribbons 50 can be made of either woven or non-woven material, such as copper foil. A pair of ribbon wires 64 may then be electrically connected to the ribbons 50, in turn connecting each heating strip 54 to the electrical cord 18. A control switch 42 may be used for controlling vibration features of the present invention.

In one embodiment, two thermostats 58 may be used, one acting as a back-up in the event of failure of the other. Connected in series with the heating strips 54, the thermostats 58 may serve to cut off power thereto in case the temperature becomes excessive. At least one current limiting control thermostat 58 may be situated within the electrical circuit of the heating strips 54 and distributed to sense whether any hot spots may be developing due to a fold, crinkle, or the like in the heating pad 10.

The radiant heating pad 10 can be made of various sizes and shapes, such as, blanket size, or of a particular shape to match the shape of a portion of a user's body. A user may comprise a living being; an animal; a mammal; a bird; a human; a pet; a dog; a cat; a horse; a cow; a single hump camel; and/or a double hump camel.

The heat generating apparatus of the present invention may comprise a blanket; an animal blanket; an equine blanket or wrap; a canine blanket; a kennel liner a bed liner; a treatment blanket adapted for use at least one of before, during, and/or after surgery; a treatment blanket adapted for use at least one of before, during, and/or after rendering of emergency and/or urgent medical treatment; a pad; a pillow; a pad adapted to heat a portion of a body comprising at least one of a heel, ankle, plantar fascia, knee, calf, hamstring, Achilles' tendon, thigh, waist, abdomen, elbow, wrist, shoulder, collar bone, temporomandibular joint, jaw, tooth, head, neck, back, hip, leg, foot, spine, loin, lung, sinus, wing, hoof, respiratory system, kidney, liver, muscle, tendon, ligament, limb, hand, chest, digit, groin, ear, eye, temple, nose, face and/or joint; clothing; footwear; a hat; a pet bed; an athletic support; a therapeutic support; a massaging support; a warming blanket; a cushioning support; a cushion; a baby changing mat, a baby mattress, an adult mattress, and/or a vibration device. Blankets, wraps, pads, and other items may be placed over the part to be treated, under the part, on the side, around, or in any useful position.

The heat generating pad 10 may comprise additives to enhance therapeutic benefits. The additives may be present in the enveloping cover 26a, via coating, impregnation, or other method. The additives may include metallic powders and/or crystals.

The metallic powders may comprise suitable materials, such as silver, including silver powder on a nanometer scale (nano-silver). Typical silver nano particles have sizes of 50-200 nanometers in equivalent diameter. Such a size provides enough specific surface area that increases contact area of silver with bacteria or viruses, essentially improving the powder's germicidal actions.

Gems, such as tourmaline, including tourmaline, may be a useful additive for the heating pad 10 layer 26a. Tourmaline may generate a weak electrical charge, enhancing the production of far infrared photon energy, negative ions, and alpha waves. The far infrared waves output by tourmaline may produce a warming effect that penetrates bodily tissues deeply and uniformly.

A mixture of tourmaline and silver powder 68, as shown in FIG. 1, may provide beneficial results. Such a mixture may allow the heating pad 10 to be used at very low temperatures emitting far infrared in the waveband of 6-25 microns (often 5-14 microns which activates the top layer of black tourmaline/nano silver powder composition 68 which in itself may amplify the electrically-stimulated far Infrared in a passive nature and reduce electromagnetic fields. The nano-silver may simultaneously create an enhanced anti-viral and anti-bacterial environment. Black Tourmaline is believed to improve circulation, strengthen the immune system, and reduce arthritic and muscle pain.

The mixture of black tourmaline and silver nano-powder 68 may be changed in percentage of mixture and or different modalities, semi-precious stones, crystals and/or metals to achieve different variations of depth and penetration and in fact wavelength of desired radiation.

It should be understood, of course, that the foregoing relates to exemplary embodiments of the invention and that modifications may be made without departing from the spirit and scope of the invention as set forth in the following claims.

We claim:

1. A heating pad, comprising:
radiant heating strips;
an electrically conductive ribbon electrically connected to the radiant heating strips;
a pair of isolator layers, each isolator layer adjacent to one side of the radiant heating strips; and
a thermally reflective layer situated between a side of the radiant heating strips and a proximal side of an enveloping cover;
wherein the enveloping cover is adapted to enclose the woven radiant heating strips, the electrically conductive ribbon, the pair of isolator layers, and the thermally reflective layer.

2. The heating pad of claim 1, wherein the enveloping cover is impregnated with metallic particles.

3. The heating pad of claim 2, wherein the enveloping cover is impregnated with silver particles.

4. The heating pad of claim 1, wherein the enveloping cover is impregnated with tourmaline.

5. The heating pad of claim 1, wherein a resistivity of the woven radiant heating strips provides a surface temperature adjacent a body to be heated which is less than or equal to about 34 degrees Celsius.

6. The heating pad of claim 1, wherein the radiant heating strips emit far infrared radiation in the wavelength range from about 5 microns to about 25 microns.

7. The heating pad of claim 6, wherein the radiant heating strips emit far infrared radiation in the wavelength range from about 6 microns to about 15 microns.

8. The heating pad of claim 1, wherein the radiant heating strips are woven radiant heating strips.

9. The heating pad of claim 1, wherein a user comprises at least one of:
a living being;
an animal;
a mammal;
a bird;
a human;
a pet;
a dog;
a cat;
a horse;
a cow;
a single hump camel; and/or
a double hump camel.

10. A heat generating apparatus comprising:
a heating pad further comprising:
woven radiant heating strips;
an electrically conductive ribbon electrically connected to the radiant heating strips;
an electrical wire adapted to transmit electrical current to the electrically conductive ribbons;
a thermally reflective layer situated between a side of the radiant heating strips and a proximal side of an enveloping cover; and
a flexible envelope of radiation permeable material enclosing the radiant heating strips and adapted such the electrical wire extends out of the flexible envelope.

11. The heating generating apparatus of claim 10, wherein the heat generating apparatus comprises at least one of:
a blanket;
an animal blanket;
an equine blanket;
a canine blanket;
a kennel liner a bed liner;
a treatment blanket adapted for use at least one of before, during, and/or after surgery;
a treatment blanket adapted for use at least one of before, during, and/or after rendering of emergency and/or urgent medical treatment;
a pad;
a pillow;
a pad adapted to heat a portion of a body comprising at least one of a heel, ankle, plantar fascia, knee, calf, hamstring, Achilles' tendon, thigh, waist, abdomen, elbow, wrist, shoulder, collar bone, temporomandibular joint, jaw, tooth, head, neck, back, hip, leg, foot, spine, loin, lung, sinus, wing, hoof, respiratory system, kidney, liver, muscle, tendon, ligament, limb, hand, chest, digit, groin, ear, eye, temple, nose, face and/or joint;
clothing;
footwear;
a hat;
a pet bed;
an equine wrap
an athletic support;
a therapeutic support;
a massaging support;
a warming blanket;
a cushioning support;
a cushion;
a vibration device;
a baby changing mat;
a baby mattress; and/or
an adult mattress.

12. The heating pad of claim 10, wherein the enveloping cover comprises metallic particles.

13. The heating pad of claim 12, wherein the enveloping cover comprises silver particles.

14. The heating pad of claim 11, wherein the enveloping cover comprises tourmaline.
